(19) United States
(12) Patent Application Publication
(10) Pub. No.: US 2021/0100360 A1
(43) Pub. Date: Apr. 8, 2021

(54) MEDICAL SUPPLY CABINET WITH A DETACHABLE LINER HAVING MEDICAL SUPPLY RETAINING POUCHES AND MACHINE READABLE CODES

(71) Applicant: Aero Healthcare, Valley Cottage, NY (US)

(72) Inventor: Timothy John Ovenden, Upper Saddle River, NJ (US)

(73) Assignee: Aero Healthcare, Valley Cottage, NY (US)

(21) Appl. No.: 17/062,044

(22) Filed: Oct. 2, 2020

Related U.S. Application Data
(60) Provisional application No. 62/912,162, filed on Oct. 8, 2019.

(51) Int. Cl. A47B 67/02; A47B 96/20; G09F 23/00; A61B 50/10

(52) U.S. Cl. CPC A47B 67/02; A47B 96/20; A61F 17/00; A61B 50/10; A61B 2050/105; G09F 23/00

(57) ABSTRACT

A medical supply cabinet includes a removable liner that attaches to the door. The removable liner, on a first side, has a plurality of pouches and associated machine readable codes or graphics to identify the particular medical supply for the particular pouch. The machine readable codes or graphics on the first side of the removable liner are located on the removable liner so as to be only visible when the associated medical supply needs replenishing. The inclusion of the associated visually machine readable codes or graphics on the first side of the removable liner provides a redundant (fail-safe) mechanism to enable an accurate identification of which medical supplies have been depleted and need replenishing.

### PRIORITY INFORMATION

The present application claims priority, under 35 U.S.C. § 119(e), from U.S. Provisional Patent Application, Ser. No. 62/912,162, filed on Oct. 8, 2019. The entire content of U.S. Provisional Patent Application, Ser. 62/912,162, filed on Oct. 8, 2019, is hereby incorporated by reference.

### BACKGROUND

A conventional medical supply cabinet 1, as illustrated in FIG. 1, includes a cabinet storage volume 3 and a door 2. The cabinet storage volume 3 includes various shelves 4, on which medical supplies can be placed and stored.

One issue with such a conventional medical supply cabinet is to know what supplies are needed for the medical supply cabinet to be properly stocked for the location that medical supply cabinet is servicing.

For example, if the medical supply cabinet needs a burn wound medical kit, an instant ice pack, an eye wound medical kit to be properly stock, in compliance with local or federal safety laws and regulations, the conventional medical supply cabinet 1 of FIG. 1 fails to have a system or mechanism to communicate what medical supplies have been exhausted and need to be replenished to bring the conventional medical supply cabinet 1 of FIG. 1 back into compliance with local or federal safety laws and regulations.

Another issue with such a conventional medical supply cabinet is that shelves are not necessarily the best apparatus to store certain medical supplies.

For example, if the conventional medical supply cabinet 1 of FIG. 1 was required to have a supply antiseptic wipes, placing the antiseptic wipes on a shelf 4 may result in the antiseptic wipes being in an orientation so that a user cannot readily recognize the antiseptic wipes without removing the antiseptic wipes from the shelf 4.

On the other hand, placing the antiseptic wipes on a shelf 4 may result in the antiseptic wipes being accidently moved to a position behind a larger unit of medical supply so that a user cannot readily see the antiseptic wipes on the shelf 4 and mistakenly order more unnecessary antiseptic wipes.

Therefore, it is desirable to provide a medical supply cabinet that effectively communicates with machine readable code what medical supplies have been exhausted and need to be replenished.

Also, it is desirable to provide a medical supply cabinet that effectively communicates with machine readable code and human discernible symbols what medical supplies have been exhausted and need to be replenished.

Additionally, it is desirable to provide a medical supply cabinet that includes a mechanism to effectively store medical supplies that are not conducive to storage on a shelf.

Furthermore, it is desirable to provide a medical supply cabinet that includes a mechanism that attaches to the door to effectively store medical supplies that are not conducive to storage on a shelf.

Moreover, it is desirable to provide a medical supply cabinet that includes a mechanism that attaches to the door to effectively store medical supplies that are not conducive to storage on a shelf and that the mechanism includes machine readable code to effectively communicates what medical supplies have been exhausted and need to be replenished.

It is further desirable to provide a medical supply cabinet that includes a mechanism that attaches to the door to effectively store medical supplies that are not conducive to storage on a shelf and that the mechanism includes machine readable code and human discernible symbols to effectively communicates what medical supplies have been exhausted and need to be replenished.

### BRIEF DESCRIPTION OF THE DRAWINGS

The drawings are only for purposes of illustrating various embodiments and are not to be construed as limiting, wherein:

FIG. 1 illustrates a conventional medical supply cabinet;

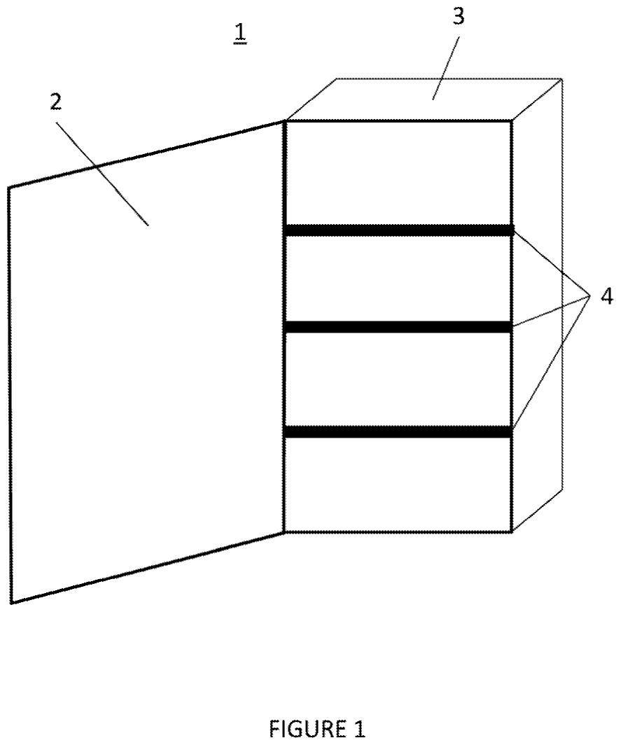

FIG. 2 illustrates an embodiment of a medical supply cabinet;

FIG. 3 illustrates an embodiment of a first side of a liner for attachment to a door of the medical supply cabinet of FIGS. 2, 5, and 7;

FIG. 4 illustrates a second side of the liner of FIG. 3;

FIG. 5 illustrates another embodiment of a medical supply cabinet;

FIG. 6 illustrates an embodiment of a second side of a liner for attachment to a door of the medical supply cabinet of FIG. 5;

FIG. 7 illustrates the medical supply cabinet of FIG. 5 having a different medical supply configuration;

FIG. 8 illustrates an embodiment of a second side of a liner for attachment to a door of the medical supply cabinet of FIG. 7;

FIG. 9 illustrates an embodiment of a second side of a liner for attachment to a door of either the medical supply cabinet of FIG. 5 or the medical supply cabinet of FIG. 7; and

FIG. 10 illustrates a further embodiment of a first side of a liner for attachment to a door of the medical supply cabinet of FIGS. 2, 5, and 7.

### DETAILED DESCRIPTION OF THE DRAWINGS

For a general understanding, reference is made to the drawings. In the drawings, like references have been used throughout to designate identical or equivalent elements. It is also noted that the drawings may not have been drawn to scale and that certain regions may have been purposely drawn disproportionately so that the features and concepts could be properly illustrated.

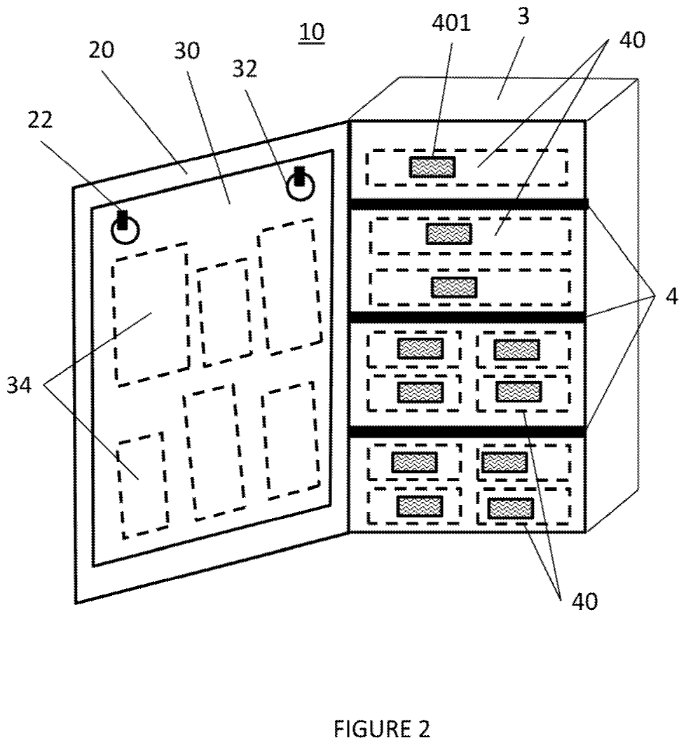

FIG. 2 illustrates a medical supply cabinet 10 that includes a cabinet storage volume 3 and a door 20. The cabinet storage volume 3 includes various shelves 4, on which medical modules 40 can be placed and stored.

As illustrated in FIG. 2, the medical modules 40 are medical modules, wherein each medical module 40 contains specific medical supplies to address a specific medical situation. For example, the medical modules 40 may be a body fluids clean-up module, a burns module, an eye wound module, a minor wound module, a resuscitation module, a serious wound module, and/or a splint & tourniquet module.

In a preferred embodiment of the medical supply cabinet, each medical module 40 has a predetermined storage location on a predetermined shelf 4. For example, the body fluids clean-up module may have the predetermined storage location of the top shelf 4 of the cabinet storage volume 3.

On the other hand, the serious wound module may have a predetermined storage location of being placed above a splint & tourniquet module on the left side of the bottom of the cabinet storage volume 3, and the burns module may have a predetermined storage location of being placed below an eye wound module on the right side of the first shelf 4 from the bottom of the cabinet storage volume 3.

Each medical module 40 also includes a radio frequency identification (RFID) tag 401 identifying the contents of the associated medical module. The medical supply cabinet 10 may have associated therewith a radio frequency identification (RFID) tag reader so that when a medical module 40 is removed from the cabinet storage volume 3, the removal of the medical module 40 can be recorded for inventory control purposes.

As illustrated in FIG. 2, the medical supply cabinet 10 also includes a removable liner 30 that attaches to the door 20. The door may include hooks 22 that engage openings 32 in the removable liner 30 to enable the attachment of the removable liner 30 to the door 20.

The removable liner 30 includes a plurality of transparent pouches 34 to store medical supplies (not shown). These medical supplies may not be conducive to storage on one of the shelves 4.

For example, the transparent pouches 34 may store triangular bandages, burn gel packets, hand sanitizer packets, gauze pads, low adherent dressing, butterfly closures, instant ice pack, visual fingertip/knuckle bandages, visual strips, visual tape, splinter probes, disposable face shield, finger cot, triple antibiotic ointment, eyewash, antiseptic wipes, forceps, first aid guide pamphlet, and/or scissors.

In a preferred embodiment of the medical supply cabinet, each transparent pouch 34 is configured to store a particular medical supply. For example, the transparent pouch 34, in the upper right corner, may be configured to store the first aid guide pamphlet. On the other hand, the transparent pouch 34, in the lower left corner, may be configured to store the antiseptic wipes.

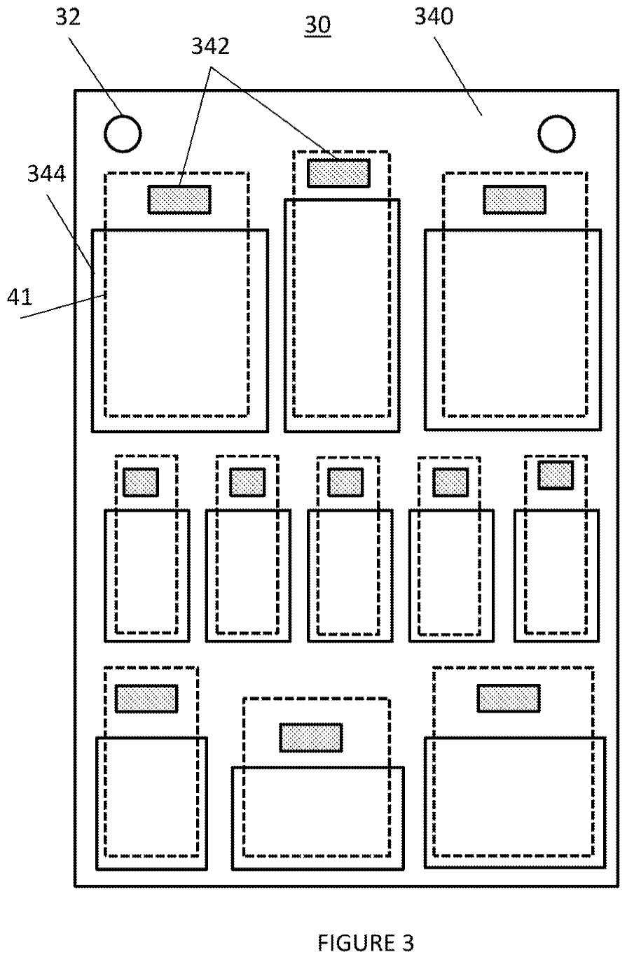

FIG. 3 illustrates a first side 340 of removable liner 30 for the medical supply cabinets of FIGS. 2, 5, and 7. As illustrated in FIG. 3, the first side 340 of removable liner 30 includes a plurality of transparent pouches 344. Each transparent pouch 344 is configured to store a particular medical supply 41.

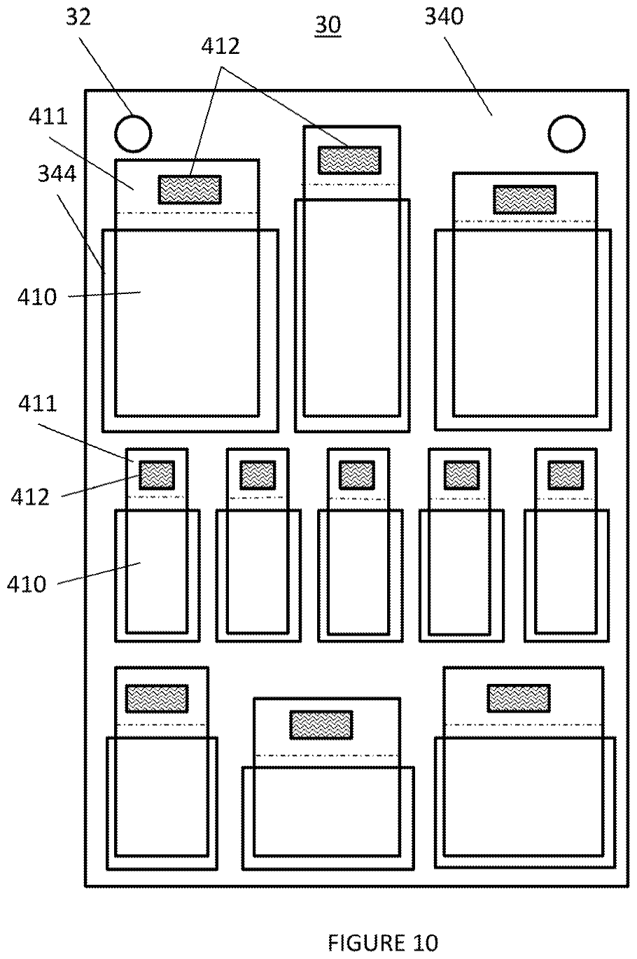

As illustrated in FIG. 10, the packaging 410 of each medical supply 41 includes a tear-away portion 411 that is detached from the packaging 410 when a user is accessing the medical supply 41 therein. The tear-away portion 411 includes a radio frequency identification (RFID) tag 412 identifying the contents of the associated packaging 410 of the medical supply 41 for inventory control purposes.

The medical supply cabinet 10 may have associated therewith a radio frequency identification (RFID) tag reader so that when a tear-away portion 411 is removed from the removable liner 30, the removal of the tear-away portion 411 can be recorded for inventory control purposes.

As further illustrated by FIG. 3, associated with each transparent pouch 344 is a machine readable code or graphic 342, located on the removable liner 30, that represents the particular medical supply 41, for which the transparent pouch 344 was configured.

As illustrated in FIG. 3, the machine readable code or graphic 342 is not visible when the associated medical supply 41 is in the transparent pouch 344. More specifically, the machine readable code or graphic 342 is hidden behind the associated medical supply 41 in the transparent pouch 344 so that machine readable code or graphic 342 cannot be read.

The machine readable code or graphic 342 may be a barcode, glyphs, a QR Code, a Unique Item Identifier code, or a set of symbols or graphical marks, that communicates to a machine (such as a scanner) the identity of the particular medical supply 41 for which the associated transparent pouch 344 was configured.

By utilizing a machine readable code or graphic that requires a visual scanning (such as carried out by a laser driven or light driven scanner) of the machine readable code or graphic 342 to effectively communicate to a machine the identity of the particular medical supply 41 for which the associated transparent pouch 344 was configured, the placement of the machine readable code or graphic 342 to be optically hidden by the medical supply 41 prevents a mistaken reading of the machine readable code or graphic 342 when the associated transparent pouch 344 still contained the particular medical supply 41 for which the associated transparent pouch 344.

Moreover, the machine readable code or graphic 342, and the location thereof, allows the full scanning of the first side 340 of removable liner 30 to determine which particular medical supplies 41 need replenishing without manually removing unnecessary scanned information because the relationship between of the location of the machine readable code or graphic 342 and the presence of particular medical supplies 41 effectively filters or blocks the scanning of unnecessary or undesired machine readable codes or graphics 342.

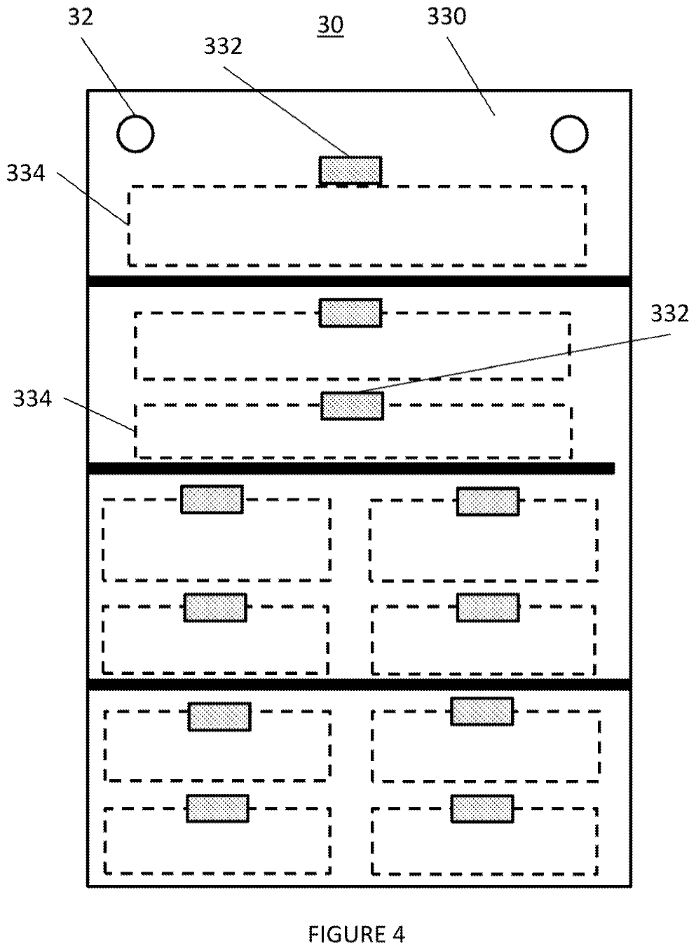

FIG. 4 illustrates a second side 330 of the removable liner 30 for the medical supply cabinets of FIG. 2. As illustrated in FIG. 4, the second side 330 of removable liner 30 includes a plurality of machine readable codes or graphics 332. Each machine readable code or graphic 332 is located on the removable liner 30 so as to represent a location of a particular medical module 40 in the cabinet storage volume 3.

Moreover, each machine readable code or graphic 332 has associated therewith human discernable symbols or graphics to effectively convey the identity of the particular medical module 40 associated therewith.

As illustrated in FIG. 4, when the removable liner 30 is attached to the door 20 so that the second side 330 of the removable liner 30 faces outwardly (away from the door 20), a comparison of the actual inventory of medical modules 40 in the cabinet storage volume 3 with a desired inventory of medical modules 40 in the cabinet storage volume 3 can be readily realized.

Thus, when determining which medical modules 40 need to be replenish, the proper machine readable code or graphic 332 can be scanned by a visual inspection of the second side 330 of the removable liner 30 and the actual inventory of medical modules 40 in the cabinet storage volume 3.

For example, if the body fluids clean-up module is missing from the top shelf 4 of the cabinet storage volume 3, the machine readable code or graphic 332 located at the top of the second side 330 of the removable liner 30 can be scanned to effectively communicate that the medical supply cabinet needs a body fluids clean-up module.

Unlike the first side 340 of the removable liner 30, the second side 330 of the removable liner 30 cannot be fully scanned without manually removing the unnecessary or undesired information from the incorrectly scanned machine readable codes or graphics 332. The second side 330 of the removable liner 30 can be effectively fully scanned if the cabinet storage volume 3 is completely empty.

The machine readable code or graphic 332 may be a barcode, glyphs, a QR Code, a Unique Item Identifier code, or a set of symbols or graphical marks, that communicates to a machine (such as a scanner) the identity of the particular medical module 40.

The machine readable code or graphic 332, and the location thereof, allows the discrete scanning of the second side 330 of removable liner 30 to determine which particular medical modules 40 need replenishing because the relationship between of the location of the machine readable code or graphic 332 and the absence of the particular medical module 40 in the cabinet storage volume 3 effectively communicates to the user of the scanner which machine readable codes or graphics 332 to scan.

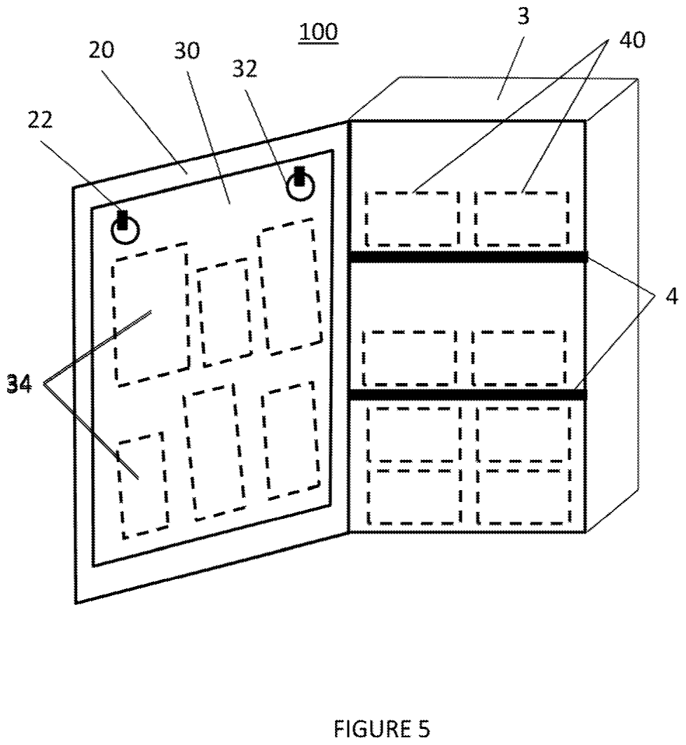

FIG. 5 illustrates a medical supply cabinet 100 that includes a cabinet storage volume 3 and a door 20. The cabinet storage volume 3 includes various shelves 4, on which medical modules 40 can be placed and stored.

As illustrated in FIG. 5, the medical modules 40 are medical modules, wherein each medical module 40 contains specific medical supplies to address a specific medical situation. For example, the medical modules 40 may be a body fluids clean-up module, a burns module, an eye wound module, a minor wound module, a resuscitation module, a serious wound module, and/or a splint & tourniquet module.

In a preferred embodiment of the medical supply cabinet, each medical module 40 has a predetermined storage location on a predetermined shelf 4. For example, the body fluids clean-up module may have the predetermined storage location of the left side of the top shelf 4 of the cabinet storage volume 3. On the other hand, the serious wound module may have a predetermined storage location of being placed above a splint & tourniquet module on the left side of the bottom of the cabinet storage volume 3, and the burns module may have a predetermined storage location of being placed on the right side of the first shelf 4 from the bottom of the cabinet storage volume 3.

As illustrated in FIG. 5, the medical supply cabinet 10 also includes a removable liner 30 that attaches to the door 20. The door may include hooks 22 that engage openings 32 in the removable liner 30 to enable the attachment of the removable liner 30 to the door 20.

The removable liner 30 includes a plurality of transparent pouches 34 to store medical supplies (not shown). These medical supplies may not be conducive to storage on one of the shelves 4.

For example, the transparent pouches 34 may store triangular bandages, burn gel packets, hand sanitizer packets, gauze pads, low adherent dressing, butterfly closures, instant ice pack, visual fingertip/knuckle bandages, visual strips, visual tape, splinter probes, disposable face shield, finger cot, triple antibiotic ointment, eyewash, antiseptic wipes, forceps, first aid guide pamphlet, and/or scissors.

In a preferred embodiment of the medical supply cabinet, each transparent pouch 34 is configured to store a particular medical supply. For example, the transparent pouch 34, in the upper right corner, may be configured to store the first aid guide pamphlet. On the other hand, the transparent pouch 34, in the lower left corner, may be configured to store the antiseptic wipes.

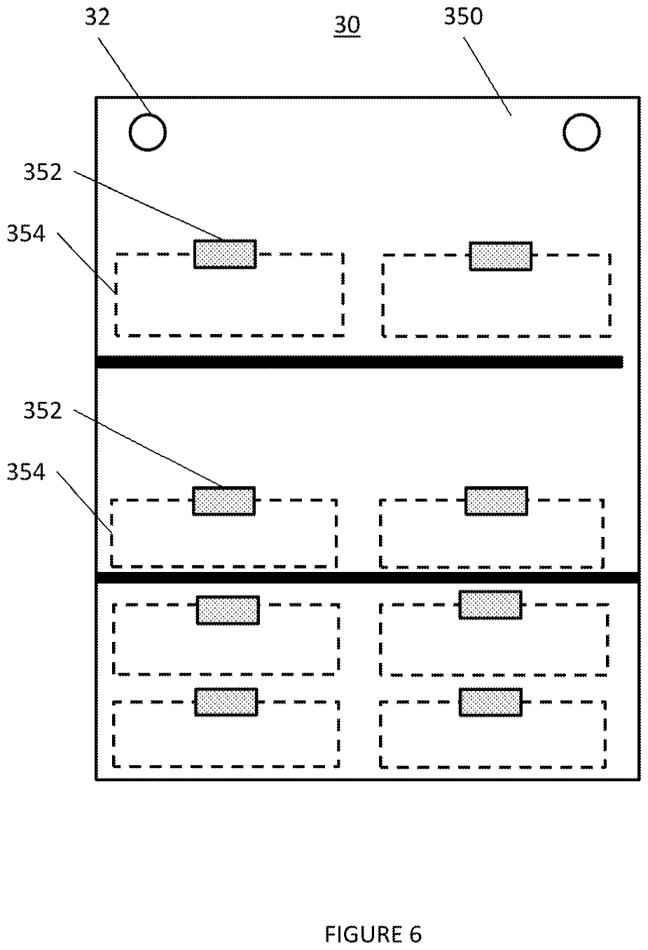

FIG. 6 illustrates a second side 350 of the removable liner 30 for the medical supply cabinet of FIG. 5. As illustrated in FIG. 6, the second side 350 of removable liner 30 includes a plurality of machine readable codes or graphics 352. Each machine readable code or graphic 352 is located on the removable liner 30 so as to represent a location of a particular medical module 40 in the cabinet storage volume 3 of FIG. 5.

Moreover, each machine readable code or graphic 352 has associated therewith human discernable symbols or graphics 354 to effectively convey the identity of the particular medical module 40 associated therewith.

As illustrated in FIG. 6, when the removable liner 30 is attached to the door 20 so that the second side 350 of the removable liner 30 faces outwardly (away from the door 20), a comparison of the actual inventory of medical modules 40 in the cabinet storage volume 3 of FIG. 5 with a desired inventory of medical modules 40 in the cabinet storage volume 3 of FIG. 5 can be readily realized.

Thus, when determining which medical modules 40 need to be replenish, the proper machine readable code or graphic 352 can be scanned by a visual inspection of the second side 350 of the removable liner 30 and the actual inventory of medical modules 40 in the cabinet storage volume 3.

For example, if the body fluids clean-up module is missing from the top shelf 4 of the cabinet storage volume 3, the machine readable code or graphic 332 located at the top left of the second side 350 of the removable liner 30 can be scanned to effectively communicate that the medical supply cabinet needs a body fluids clean-up module.

Unlike the first side 340 of the removable liner 30, the second side 350 of the removable liner 30 cannot be fully scanned without manually removing the unnecessary or undesired information from the incorrectly scanned machine readable codes or graphics 352. The second side 350 of the removable liner 30 can be effectively fully scanned if the cabinet storage volume 3 is completely empty.

The machine readable code or graphic 352 may be a barcode, glyphs, a QR Code, a Unique Item Identifier code, or a set of symbols or graphical marks, that communicates to a machine (such as a scanner) the identity of the particular medical module 40.

The machine readable code or graphic 352, and the location thereof, allows the discrete scanning of the second side 350 of removable liner 30 to determine which particular medical modules 40 need replenishing because the relationship between of the location of the machine readable code or graphic 352 and the absence of the particular medical module 40 in the cabinet storage volume 3 effectively communicates to the user of the scanner which machine readable codes or graphics 352 to scan.

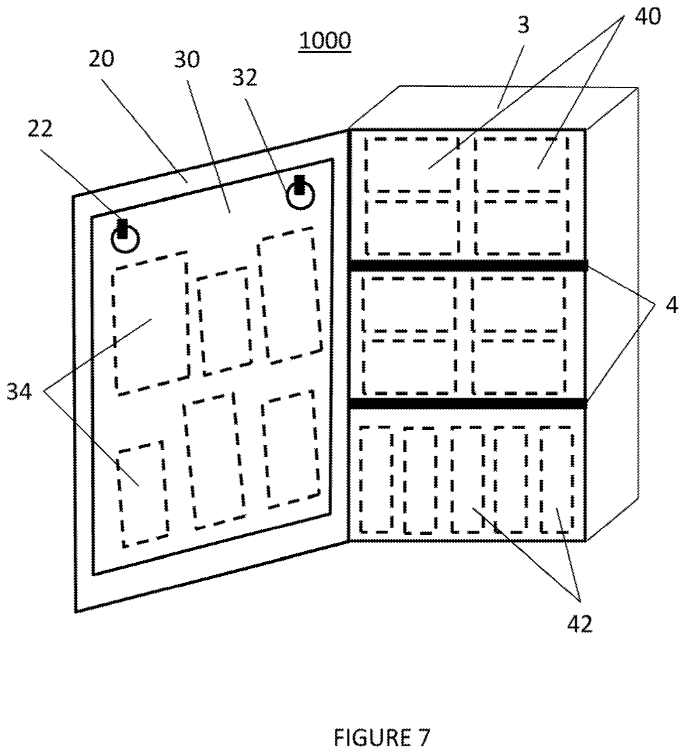

FIG. 7 illustrates a medical supply cabinet 1000 that includes a cabinet storage volume 3 and a door 20. The cabinet storage volume 3 includes various shelves 4, on which medical modules 40 can be placed and stored. The cabinet storage volume 3 includes containers of medicine 42 located on the bottom of the cabinet storage volume 3.

As illustrated in FIG. 7, the medical modules 40 are medical modules, wherein each medical module 40 contains specific medical supplies to address a specific medical situation. For example, the medical modules 40 may be a body fluids clean-up module, a burns module, an eye wound module, a minor wound module, a resuscitation module, a serious wound module, and/or a splint & tourniquet module.

Each container of medicine 42 contains a specific medicine. For example, a container of medicine 42 may contain pain reliever, ibuprofen, non-aspirin, cold relief, and/or antacid.

In a preferred embodiment of the medical supply cabinet, each medical module 40 has a predetermined storage location on a predetermined shelf 4. For example, the body fluids clean-up module may have the predetermined storage location above a splint & tourniquet module on the left side of the top shelf 4 of the cabinet storage volume 3. On the other hand, the serious wound module may have a predetermined storage location of being placed above a minor module on the right side of the first shelf 4 from the bottom of the cabinet storage volume 3.

On the bottom of the cabinet storage volume 3, each container of medicine 42 has a predetermined storage location. For example, a pain reliever container may be located on the left side of bottom of the cabinet storage volume 3.

As illustrated in FIG. 7, the medical supply cabinet 10 also includes a removable liner 30 that attaches to the door 20. The door may include hooks 22 that engage openings 32 in the removable liner 30 to enable the attachment of the removable liner 30 to the door 20.

The removable liner 30 includes a plurality of transparent pouches 34 to store medical supplies (not shown). These medical supplies may not be conducive to storage on one of the shelves 4.

For example, the transparent pouches 34 may store triangular bandages, burn gel packets, hand sanitizer packets, gauze pads, low adherent dressing, butterfly closures, instant ice pack, visual fingertip/knuckle bandages, visual strips, visual tape, splinter probes, disposable face shield, finger cot, triple antibiotic ointment, eyewash, antiseptic wipes, forceps, first aid guide pamphlet, and/or scissors.

In a preferred embodiment of the medical supply cabinet, each transparent pouch 34 is configured to store a particular medical supply. For example, the transparent pouch 34, in the upper right corner, may be configured to store the first aid guide pamphlet. On the other hand, the transparent pouch 34, in the lower left corner, may be configured to store the antiseptic wipes.

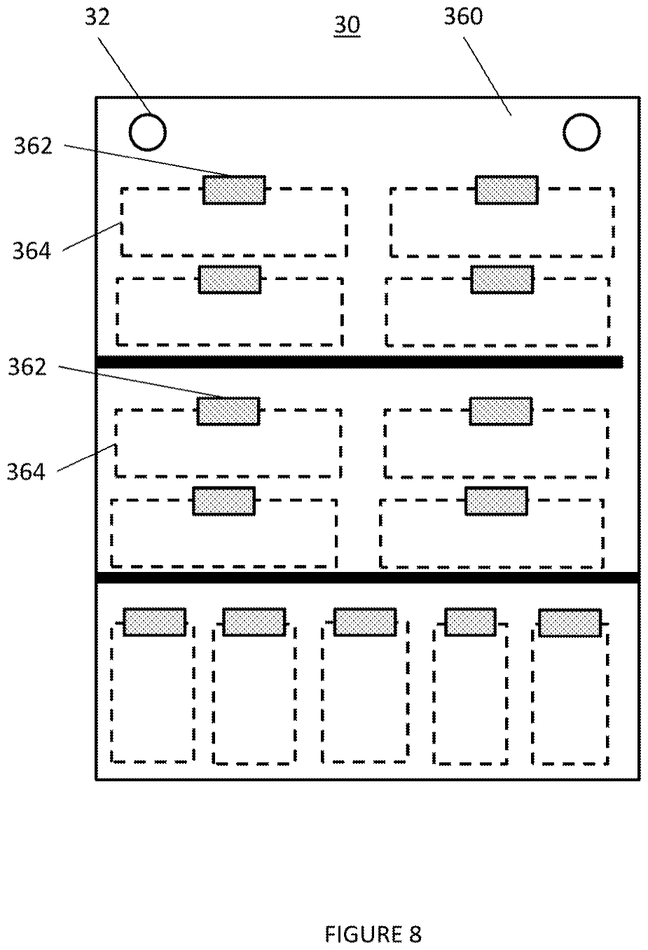

FIG. 8 illustrates a second side 360 of the removable liner 30 for the medical supply cabinet of FIG. 7. As illustrated in FIG. 8, the second side 360 of removable liner 30 includes a plurality of machine readable codes or graphics 362. Each machine readable code or graphic 362 is located on the removable liner 30 so as to represent a location of a particular medical module 40 and a container of medicine 42 in the cabinet storage volume 3 of FIG. 7.

Moreover, each machine readable code or graphic 362 has associated therewith human discernable symbols or graphics 364 to effectively convey the identity of the particular medical module 40 associated therewith.

As illustrated in FIG. 8, when the removable liner 30 is attached to the door 20 so that the second side 360 of the removable liner 30 faces outwardly (away from the door 20), a comparison of the actual inventory of medical modules 40 in the cabinet storage volume 3 of FIG. 7 with a desired inventory of medical modules 40 in the cabinet storage volume 3 of FIG. 7 can be readily realized.

Thus, when determining which medical modules 40 need to be replenish, the proper machine readable code or graphic 362 can be scanned by a visual inspection of the second side 360 of the removable liner 30 and the actual inventory of medical modules 40 in the cabinet storage volume 3.

For example, if the body fluids clean-up module is missing from the top shelf 4 of the cabinet storage volume 3, the machine readable code or graphic 362 located at the top left of the second side 360 of the removable liner 30 can be scanned to effectively communicate that the medical supply cabinet needs a body fluids clean-up module.

Unlike the first side 340 of the removable liner 30, the second side 360 of the removable liner 30 cannot be fully scanned without manually removing the unnecessary or undesired information from the incorrectly scanned machine readable codes or graphics 362. The second side 360 of the removable liner 30 can be effectively fully scanned if the cabinet storage volume 3 is completely empty.

The machine readable code or graphic 362 may be a barcode, glyphs, a QR Code, a Unique Item Identifier code, or a set of symbols or graphical marks, that communicates to a machine (such as a scanner) the identity of the particular medical module 40.

The machine readable code or graphic 362, and the location thereof, allows the discrete scanning of the second side 360 of removable liner 30 to determine which particular medical modules 40 need replenishing because the relationship between of the location of the machine readable code or graphic 362 and the absence of the particular medical module 40 in the cabinet storage volume 3 effectively communicates to the user of the scanner which machine readable codes or graphics 362 to scan.

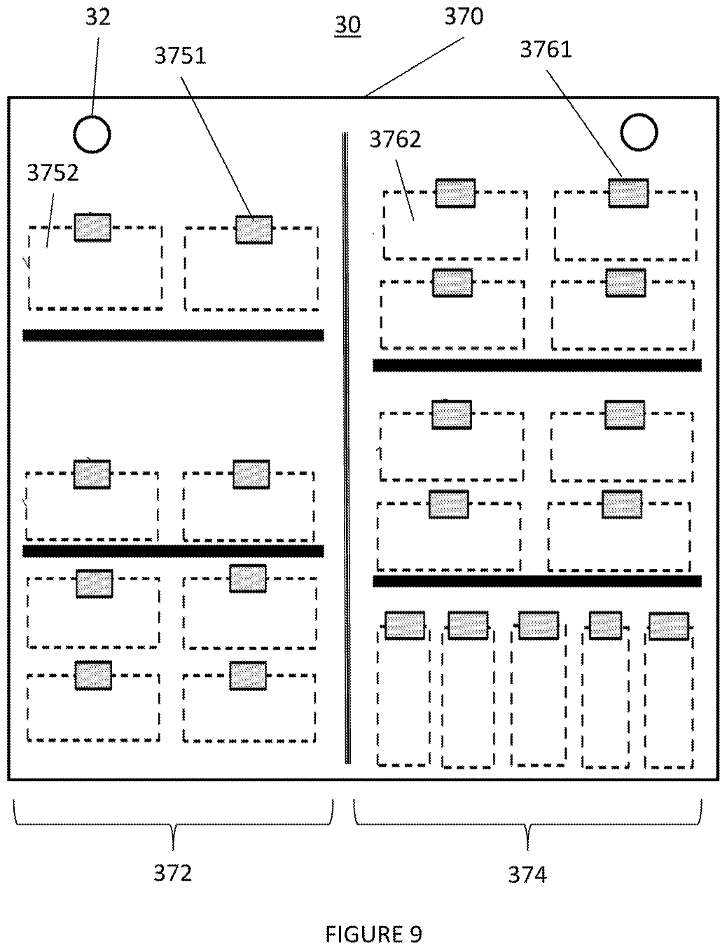

FIG. 9 illustrates a second side 370 of the removable liner 30 for both the medical supply cabinet of FIG. 5 and the medical supply cabinet of FIG. 7. As illustrated in FIG. 9, on one side (left side) of the second side 370 of removable liner 30 includes a plurality of machine readable codes or graphics 3751 and on the other side (right side) of the second side 370 of removable liner 30 includes a plurality of machine readable codes or graphics 3761.

Each machine readable code or graphic 3761 is located on the removable liner 30 so as to represent a location of a particular medical module 40 and a container of medicine 42 in the cabinet storage volume 3 of FIG. 7.

Each machine readable code or graphic 3751 is located on the removable liner 30 so as to represent a location of a particular medical module 40 in the cabinet storage volume 3 of FIG. 5.

Moreover, each machine readable code or graphic 3761 has associated therewith human discernable symbols or graphics 3762 to effectively convey the identity of the particular medical module 40 or container of medicine 42 associated therewith.

Each machine readable code or graphic 3751 has associated therewith human discernable symbols or graphics 3752 to effectively convey the identity of the particular medical module 40 associated therewith.

As illustrated in FIG. 9, when the removable liner 30 is attached to the door 20 so that the second side 370 of the removable liner 30 faces outwardly (away from the door 20), a comparison of the actual inventory of medical modules 40 in the cabinet storage volume 3 of FIG. 7 or the actual inventory of medical modules 40 in the cabinet storage volume 3 of FIG. 5 with a desired inventory of medical modules 40 in the cabinet storage volume 3 of FIG. 7 or a desired inventory of medical modules 40 in the cabinet storage volume 3 of FIG. 5 can be readily realized.

Thus, when determining which medical modules 40 need to be replenish, the proper machine readable code or graphic (3751 or 3761) can be scanned by a visual inspection of the second side 370 of the removable liner 30 and the actual inventory of medical modules 40 (and/or containers of medicine 42) in the appropriate cabinet storage volume 3.

For example, if the body fluids clean-up module is missing from the top shelf 4 of the cabinet storage volume 3 of FIG. 5, the machine readable code or graphic 3752 located at the top left of the second side 370 of the removable liner 30 can be scanned to effectively communicate that the medical supply cabinet needs a body fluids clean-up module.

Unlike the first side 340 of the removable liner 30, the second side 370 of the removable liner 30 cannot be fully scanned without manually removing the unnecessary or undesired information from the incorrectly scanned machine readable codes or graphics (3751 or 3761).

The machine readable code or graphic (3751 or 3761) may be a barcode, glyphs, a QR Code, a Unique Item Identifier code, or a set of symbols or graphical marks, that communicates to a machine (such as a scanner) the identity of the particular medical module 40 and/or container of medicine 42.

The machine readable code or graphic (3751 or 3761), and the location thereof, allows the discrete scanning of the second side 370 of removable liner 30 to determine which particular medical modules 40 and/or containers of medicine 42 need replenishing because the relationship between of the location of the machine readable code or graphic (3751 or 3761) and the absence of the particular medical module 40 and/or containers of medicine 42 in the cabinet storage volume 3 effectively communicates to the user of the scanner which machine readable codes or graphics (3751 or 3761) to scan.

As described above, the removable liner and associated visually machine readable codes or graphics allows the conventional medical supply cabinet to not only increase the amount of supplies contained therein, but enables an accurate replenishing of the used medical supplies.

By exposing the visually machine readable codes or graphics after the medical supply on the removable liner is used (the packaging for the medical supply is discarded), a user can use a machine readable code scanner to accurately identify, even though the packaging of the depleted medical supply is no longer in the transparent pouch, which medical supplies have been depleted and need replenishing.

As an enhancement to this system, the inclusion of RFID tags in the tear away portion of the packaging can also assist in monitoring inventory by recording the use of the medical supplies for inventory control purposes.

However, the use of a RFID tag requires the user of the medical supply to actively scan the tear-away portion of the packaging. In emergency situations, there may not be time to scan the tear-away portion of the packaging or the tear-away portion of the packaging is discarded before a scanning can take place. Thus, the inclusion of the associated visually machine readable codes or graphics on the removable liner provides a redundant (fail-safe) mechanism to enable an accurate identification of which medical supplies have been depleted and need replenishing.

It is noted that although the various embodiments described above use visually machine readable codes or graphics, the machine readable code or graphic may be a RFID tag to identify the medical module, medical supply, and/or container of medicine.

It is further noted that although the various embodiments described above use transparent pouches on the removable liner, the pouches may be non-transparent so long as the pouches do not visually impede the visually machine readable codes or graphics.

In summary, a medical supply cabinet includes a removable liner that attaches to the door. The removable liner, on a first side, has a plurality of pouches and associated machine readable codes or graphics to identify the particular medical supply for the particular pouch. The machine readable codes or graphics on the first side of the removable liner are located on the removable liner so as to be only visible when the associated medical supply needs replenishing.

The inclusion of the associated visually machine readable codes or graphics on the first side of the removable liner provides a redundant (fail-safe) mechanism to enable an accurate identification of which medical supplies have been depleted and need replenishing.

The removable liner, on a second side, has a plurality of machine readable codes or graphics associated with medical modules and/or containers of medicine located in the cabinet storage volume. The machine readable codes or graphics on the second side of the removable liner are located on the removable liner so as to be associated with the locations of medical modules and/or containers of medicine in the cabinet storage volume.

The inclusion of the associated visually machine readable codes or graphics on the second side of the removable liner provides a redundant (fail-safe) mechanism to enable an accurate identification of which medical modules have been depleted and need replenishing.

It will be appreciated that variations of the above-disclosed embodiments and other features and functions, or alternatives thereof, may be desirably combined into many other different systems or applications. Also, various presently unforeseen or unanticipated alternatives, modifications, variations, or improvements therein may be subsequently made by those skilled in the art which are also intended to be encompassed by the description above.

What is claimed is:

1. A medical supply cabinet, comprising:
a door;
a removable liner attachable to said door;
said removable liner, on a first side, having a plurality of pouches and associated machine readable codes to identify a medical supply associated with a pouch;
said machine readable codes on said first side of said removable liner being located on said removable liner so as to be only visible when the medical supply is removed from the associated pouch to provide a redundant mechanism to enable accurate identification of which medical supplies have been depleted.

2. The medical supply cabinet, as claimed in claim 1, wherein said removable liner, on a second side, having a plurality of machine readable codes associated with medical containers located within a volume of the medical supply cabinet to provide a redundant mechanism to enable accurate identification of which medical containers have been depleted.

3. A medical supply cabinet, comprising:
a door;
a removable liner attachable to said door;
said removable liner, on a first side, having a plurality of pouches and associated graphics to identify a medical supply associated with a pouch;
said graphics on said first side of said removable liner being located on said removable liner so as to be only visible when the medical supply is removed from the associated pouch.

4. The medical supply cabinet, as claimed in claim 2, wherein said removable liner, on a second side, having a plurality of graphic associated with medical containers located within a volume of the medical supply cabinet to provide a redundant mechanism to enable accurate identification of which medical containers have been depleted.
